(19) United States
(12) Patent Application Publication
(10) Pub. No.: US 2005/0129691 A1
(43) Pub. Date: Jun. 16, 2005

(54) Method for treating anxiety and mood disorders in older subjects

(75) Inventor: Robert Thomas Gerlai, Carmel, IN (US)

Correspondence Address: ELI LILLY AND COMPANY;PATENT DIVISION, P.O. BOX 6288, INDIANAPOLIS, IN, 46206-6288, US

(73) Assignee: Eli Lily and Company Patent Division, Indianapolis, IN (US)

(21) Appl. No.: 10/512,527

(22) PCT Filed: Apr. 17, 2003
(86) PCT No.: PCT/US03/10473

Related U.S. Application Data
(60) Provisional application No. 60/375,462, filed on Apr. 25, 2002.

(51) Int. Cl. A61K 39/395

(52) U.S. Cl. 424/145.1

(57) ABSTRACT

The present invention is a method for treating anxiety or mood disorders in elderly subjects comprising administering to the subject exhibiting an anxiety or mood disorder an effective amount of an agent that modulates Aβ in the subject.

This invention relates to methods of treating certain mental disorders in elderly subjects.

Millions of older people—indeed, the majority—cope constructively with the physical limitations, cognitive changes, and various losses, such as bereavement, that frequently are associated with later life. The capacity for sound mental health among older adults notwithstanding, a substantial proportion of the population 55 and older—almost 20 percent of this age group—experience specific mental disorders that are not part of “normal” aging. The data below represent the 1-year prevalence (%) of various mental disorders among Americans above age 55. In the same study, the prevalence of any mental disorder was 19.8% and the prevalence of severe cognitive impairment was 6.6%.

| | % | | % |
|---|---|---|---|
| any anxiety disorder | 11.4 | any mood disorder | 4.4 |
| simple phobia | 7.3 | major depressive episode | 3.8 |
| social phobia | 1.0 | unipolar major depression | 3.7 |
| agoraphobia | 4.1 | dysthymia | 1.6 |
| panic disorder | 0.5 | schizophrenia | 0.6 |
| obsessive-compulsive disorder | 1.5 | other | 0.6 |

Depression in older adults not only causes distress and suffering but also causes impairments in physical, mental, and social functioning, and increased mortality, especially from suicide, heart disease and possibly cancer. Estimates of the prevalence of major depression and its association with age and other factors vary widely, depending on the definition and the procedure used for counting persons with depression. The prevalence of major depression is thought to decline with age, while depressive symptoms increase (symptoms that now might warrant classification as minor depression). Older people often present several depressive symptoms together, a condition often referred to as “minor depression,” that can be as disabling as major depression. Minor depression, despite the implications of the term, is major in its prevalence and impact. Eight to 20 percent of older adults and up to 37 percent in primary care settings suffer from depressive symptoms. In late life, the course of depression tends to be more chronic than that in younger adults with longer recurrent episodes punctuated by shorter remissions, and highly variable response to treatment. Response rates to treatment are thought to be somewhat successful (between 60 and 80 percent), but the response generally takes longer than that for younger adults.

The most serious consequence of depression in later life—especially untreated or inadequately treated depression—is increased mortality from either suicide or somatic illness. Older persons (65 years and above) have the highest suicide rates of any age group. Suicide in older adults is most associated with late-onset depression. Among patients 75 years of age and older, 60 to 75 percent of suicides have diagnosable depression.

Depression in the elderly leads to increased mortality from other diseases, such as heart disease and cancer. In the case of myocardial infarction, depression elevates mortality risk fivefold. Chronic depression (lasting an average of about 4 years) raises the risk of cancer by 88 percent in older people.

Late-life depression is particularly costly because of the excess disability that it causes and its deleterious interaction with physical health. Older primary care patients with depression visit the doctor and emergency room more often, use more medication, incur higher outpatient charges, and stay longer at the hospital.

Known pharmaceutical agents for treating depression in the elderly vary in their effectiveness, and all suffer from side effects that are especially worrisome in this population. Tricyclic antidepressants (TCAs), for example, have been widely used to treat elderly depressed patients, but anticholinergic effects such as dry mouth, urinary retention, and constipation lead to severe problems in older adults, such as bowel impaction due to persistent constipation or prevention of the wearing of dentures because of dry mouth. The anticholinergic effects of the TCAs may also cause tachycardia or arrhythmias and can further compromise preexisting cardiac disease. Central anticholinergic effects may result in acute confusional states or memory problems in the depressed older adult. Orthostatic hypotension, which may lead to falls and hip fractures, is also a concern when the TCAs are administered.

Selective serotonin reuptake inhibitors (SSRIs) have fewer anticholinergic and cardiovascular side effects than the TCAs, but this is counterbalanced by a significant potential for drug-drug interactions. This is of clinical importance since older adults commonly receive a large number of medications. The SSRIs vary in their inhibition of the cytochrome p450 family of isoenzymes. Newer non-SSRI antidepressants are often suggested for treating later life depression because their side effects are better tolerated by older adults.

Clinical use of monoamine oxidase inhibitors is often restricted to patients who are refractory to other antidepressant drugs because of potentially life-threatening pharmacodynamic interactions with sympathomimetic drugs or tyramine-containing foods and beverages. The sympathomimetic amines (e.g., phenylpropanolamine and pseudoephedrine) may be present in over-the-counter decongestant products that older patients are prone to self-administer. An additional concern is the risk of orthostatic hypotension, which occurs even at therapeutic doses. Bupropion, though generally well tolerated, requires added caution because of an increased risk of seizures.

Information about the course and treatment of anxiety lags behind that of other common mental conditions in the elderly, such as depression and Alzheimer's. Anxiety is at least as common in the old as in the young, although how and when it appears is distinctly different in older adults.

Overall, community-based prevalence estimates indicate that about 11.4 percent of adults aged 55 years and older meet criteria for an anxiety disorder in 1 year. Phobic anxiety disorders are among the most common mental disturbances in late life. Anxiety symptoms that do not fulfill the criteria for specific syndromes are reported in as many as 17 percent of older men and 21 percent of older women.

Drugs used to treat anxiety disorders overlap significantly with those used to treat depression, and include selective serotonin reuptake inhibitors (SSRIs), tricyclic antidepressants, benzodiazepines, beta blockers, and monoamine oxidase inhibitors (MAOIs). Benzodiazepines are marginally effective at best in treating chronic anxiety in older patients. The half-life of certain benzodiazepines and their metabolites may be significantly extended in older patients (particularly for the compounds with long half-life). If taken over extended periods, even short-acting benzodiazepines tend to accumulate in older individuals. Thus, it is generally recommended that any use of benzodiazepines be limited to discrete periods (less than 6 months) and that long-acting compounds be avoided in this population. Side effects of benzodiazepines may include drowsiness, fatigue, psychomotor impairment, memory or other cognitive impairment, confusion, paradoxical reactions, depression, respiratory problems, abuse or dependence problems, and withdrawal reactions. Benzodiazepine toxicity in older patients includes sedation, cerebellar impairment (manifested by ataxia, dysarthria, incoordination, or unsteadiness), cognitive impairment, and psychomotor impairment. Psychomotor impairment from benzodiazepines can have severe consequences, leading to impaired driver skills, motor vehicle crashes, and falls.

Buspirone, an anxiolytic (antianxiety) agent that is chemically and pharmacologically distinct from benzodiazepines may require up to 4 weeks to take effect, and significant adverse reactions to buspirone are found in 20 to 30 percent of anxious older patients, including most frequently, gastrointestinal symptoms, dizziness, headache, sleep disturbance, nausea/vomiting, uneasiness, fatigue, and diarrhea.

Thus, it can be concluded from this brief survey of anxiety and mood disorders in older persons that these are serious and costly conditions for which current pharmaceutical agents provide varying degrees of effectiveness, but often with risky adverse reactions and a real possibility of adverse interactions with the many other agents that older people commonly use for other ailments. There is an increasing need as many populations age for new pharmaceutical agents that are effective in treating anxiety and mood disorders in the elderly, and that are more compatible with the many other medications that the elderly commonly receive for other diseases and conditions.

Alzheimer's disease (AD), a disorder of pivotal importance to older adults, strikes 8 to 15 percent of people over the age of 65. Alzheimer's disease is one of the most feared mental disorders because of its gradual, yet relentless, attack on memory. Memory loss, however, is not the only impairment. Symptoms extend to other cognitive deficits in language, object recognition, and executive functioning.

Behavioral symptoms—such as psychosis, agitation, depression, and wandering—are common and impose tremendous strain on caregivers. Of the behavioral symptoms experienced by patients with Alzheimer's disease, depression and anxiety occur most frequently during the early stages, while psychoses occur later. Though behavioral symptoms have received less attention than cognitive symptoms, they have serious ramifications, such as, patient and caregiver distress, premature institutionalization, and significant compromise of the quality of life of patients and their families. Alzheimer's disease, especially its behavioral symptoms, appears to place patients at risk for abuse by caregivers. Forty to fifty percent of Alzheimer's patients have symptoms of depression and the depression accelerates loss of functioning in everyday activities. Depression in Alzheimer's is different from other depressive disorders [Olin, et al., Am. J. Geriatr. Psychiatry 10:125-128 and 129-141 (2002)]. Even modest reduction in behavioral symptoms can produce substantial improvements in functioning and quality of life.

New therapies are being studied for their ability to ameliorate or modify the significant memory loss that is characteristic of AD. Among them, lowering the levels of the Aβ peptide in the brain has been proposed to ameliorate memory loss and improve cognitive abilities in animal models of AD, and development of pharmaceutical agents to reduce Aβ is in progress. Among the pharmaceutical approaches being studied for ameliorating the effects of Aβ is the use of antibodies that bind Aβ peptide.

No link between the anxiety disorders and mood disorders discussed above and the Aβ peptide is known. Consequently, drugs potentially ameliorating Aβ-related conditions and pathologies have not been tested with regard to their effects on behavior, other than those associated with cognitive abilities.

I quite surprisingly found that when mice were injected twice within a week before fear conditioning with an antibody that binds to Aβ peptide, they exhibited significantly reduced “long-body” posture, a behavioral trait also called “stretch attend posture” in the literature. This behavior is known to be elicited in rodents by pain or fear (e.g. electric shocks, or stimuli previously associated with the shocks, or stimuli associated with natural predators of rodents). This reduction, observed in elderly (11 months old) wild type and transgenic mice that overproduce Aβ in their brains, represents reduced fear or reduced anxiety, which is likely to also affect mood. Most notably, the effect of anti-Aβ antibody was significant both in transgenic mice and in wild-type mice. In summary, I have surprisingly discovered that administering an agent that modulates levels of Aβ to aged mice reduces anxiety in the mice, regardless of their status with respect to Aβ.

### BRIEF SUMMARY OF THE INVENTION

Accordingly, the present invention is a method for treating anxiety disorders and mood disorders in an elderly subject, comprising administering to the subject an effective dose of an anti-Aβ antibody.

### DETAILED DESCRIPTION OF THE INVENTION

The term “treating” includes prophylaxis (preventing), amelioration (reducing or reversing), or elimination of a sign, symptom, condition, disease, or disorder. “Anxiety disorder” is a generic term for disorders that involve anxiety. The five major anxiety disorders are panic disorder, obsessive-compulsive disorder, post-traumatic stress disorder, generalized anxiety disorder and phobias (including social phobia, also called social anxiety disorder). Among the anxiety disorders that are treated in the practice of the present invention include are obsessive-compulsive disorder, panic disorder, panic attack, agoraphobia, post-traumatic stress disorder, social phobia, disruptive behavior disorder, and chronic fatigue syndrome.

Most of the disorders discussed here are described and categorized in the DIAGNOSTIC AND STATISTICAL MANUAL OF MENTAL DISORDERS, (4th edition, 1994), published by the American Psychiatric Association (hereinafter referred to as DSM or DSM-IV). In the discussion below, the DSM codes for the disorders will be given where appropriate.

Obsessive-compulsive disorder, DSM 300.3, is characterized by recurrent obsessions or compulsions that are severe enough to be time consuming or cause distress or impairment of the patient's life. Obsessions are persistent ideas, thoughts, impulses or images that are recognized by the patient to be intrusive and inappropriate and cause anxiety or distress. The individual senses that the obsession is alien, not under control and not the kind of thought that the patient would expect to have. Common obsessions include repeated thoughts about contamination, repeated doubts, a need to arrange things in a particular order, aggressive or horrific impulses and sexual imagery. Compulsions are repetitive behaviors, such as hand washing, or mental acts, such as counting or repeating words silently, the goal of which is to prevent or reduce anxiety or distress. By definition, compulsions are either clearly excessive or not realistically connected with that which they are designed to neutralize or prevent.

Panic attack, panic disorder and agoraphobia, categorized as DSM 300.01, 300.21 and 300.22, are characterized by irrational sense of imminent danger or doom, an urge to escape, or a fear of being in a situation from which escape might be difficult. The patient exhibits symptoms such as palpitations, accelerated heart rate, sweating, sensations of shortness of breath, chest pain, nausea, dizziness, fear of dying, and the like, and may have such attacks very frequently.

Social phobia, DSM 300.23, produces a marked and persistent fear of social or performance situations in which embarrassment may occur. Exposure to such a situation may result in a panic attack, or other anxious response. Most often, patients with the disorder simply avoid situations of the type that they dread, producing an obvious dislocation in the patient's life.

Post-traumatic stress disorder, DSM 309.81, afflicts patients following exposure to a traumatic stress involving personal experience of an event involving actual or threatened death of injury. Such traumatic events include experiences such as military combat, personal assault, kidnapping, terrorist attack, torture, natural or man-made disasters, severe accidents, or being diagnosed with a dreaded illness. Learning about such events occurring to others, particularly a family member or close friend, also may produce the disorder. Triggering events that symbolize the traumatic event, such as an anniversary, may recreate the stress and bring on the disorder long after the event is passed. Patients strive to avoid stimuli associated with the trauma, even to the point of amnesia or reduced responsiveness to other people in general.

Diagnosis of these disorders, or the identification of a patient at risk of one or more of them, is to be made by a physician or psychiatrist. It is presently believed that administration of an effective dose of an anti-Aβ agent results in the alleviation of the effects of the disorder from which the patient suffers, or even the elimination of the disorder completely. Diagnosis of anxiety disorders in the elderly may be aided by careful inquiry, as described by Lang, A. J., et al., “Anxiety Disorders: How to Recognize and Treat the Medical Symptoms of Emotional Illness,” Geriatrics 56: 24-27, 31-34 (2001). Likewise, diagnosis of depression and anxiety in Alzheimer's patients may be more challenging than in other elderly patients. Recent diagnostic criteria for depression in Alzheimer's disease will aid the diagnosis [Olin, et al., Am. J Geriatr. Psychiatry 10:125-128 and 129-141 (2002)].

“Anxiety” means the subjective unpleasant feeling of nervousness or distress in response to a feared situation (symptoms), sometimes accompanied by physiological signs including nausea, trembling, breathlessness, sweating and increased heart beat. Mental disorders characterized by felt anxiety or related symptoms are classified as “Anxiety Disorders.” The ability of an agent to treat anxiety and related disorders may be demonstrated using the techniques described hereinbelow or the well-known fear-potentiated startle and elevated plus maze models of anxiety [e.g., Davis, Psychopharmacology, 62:1 (1979); Lister, Psychopharmacology, 92: 180-185 (1987); and U.S. Pat. No. 5,750,566].

“Mood disorders” are mental disorders that involve mood, including, depression, major depressive episode, unipolar major depression, dysthymia, schizophrenia, and minor depression, late-onset depression, and traumatic grief,

“Depression” means behavioral inhibition in response to conflicting experience, that manifests as increased immobility over a prolonged period of time, or that manifests also as reduced motivation to escape punishment or work for reward. For humans, depression is a mental state characterized by extreme feeling of sadness, despair, hopelessness, low self-esteem, extremely strong and unreasonable negative feeling.

Experiencing five or more of the following symptoms each day during a two-week period or symptoms interfering with work or family activities can indicate the presence of clinical depression: prolonged sadness or unexplained crying spells; significant changes in appetite or sleep patterns; irritability, anger, worry, agitation, or anxiety; pessimism; indifference; loss of energy or persistent tiredness; feelings of guilt or worthlessness; inability to concentrate; indecisiveness; inability to take pleasure in former interests; social withdrawal; unexplained aches and pains; or recurring thoughts of death and suicide.

The term “major depression” refers to conditions with a major depressive episode, such as major depressive disorder, bipolar disorder, and related conditions. Major depressive disorder, the most common type of major depression in adults, is characterized by one or more episodes that include the following symptoms: depressed mood, loss of interest or pleasure in activities, significant weight loss or gain, sleep disturbance, psychomotor agitation or retardation, fatigue, feelings of worthlessness, loss of concentration, and recurrent thoughts of death or suicide. Major depressive disorder cannot be diagnosed if symptoms last for less than 2 months after bereavement, among other exclusionary factors (DSM-IV).

Most older patients with symptoms of depression do not meet the full criteria for major depression. The new diagnostic entity of minor depression has been proposed to characterize some of these patients. “Minor depression,” a subsyndromal form of depression, is not yet recognized as an official disorder, though DSM-IV proposes further research on it.

Minor depression is more frequent than major depression in the elderly, with 8 to 20 percent of older community residents displaying symptoms. The diagnosis of minor depression is not yet standardized; the research criteria proposed in DSM-IV are the same as those for major depression, but a diagnosis would require fewer symptoms and less impairment. Minor depression, in fact, is not thought to be a single syndrome, but rather a heterogeneous group of syndromes that may signify either an early or residual form of major depression, a chronic, though mild, form of depression that does not present with a full array of symptoms at any one time, called dysthymia, or a response to an identifiable stressor.

Major or minor depression diagnosed with first onset later than age 60 has been termed late-onset depression. Late-onset depression is not a diagnosis; rather, it refers to a subset of patients with major or minor depression whose later age at first onset imparts slightly different clinical characteristics, suggesting the possibility of distinct etiology. Late-onset depression shares many clinical characteristics with early-onset depression, yet some distinguishing features exist. Patients with late-onset depression display greater apathy and less lifetime personality dysfunction. Cognitive deficits may be more prominent, with more impaired executive and memory functioning and greater medial temporal lobe abnormalities on magnetic resonance imaging, similar to those seen in dementia.

Risk factors for late-onset depression, based on results of prospective studies, include widowhood, educational attainment less than high school, impaired functional status, and heavy alcohol consumption. Late-life mental disorders are often detected in association with somatic illness. The prevalence of clinically significant depression in later life is estimated to be highest—approximately 25 percent—among those with chronic illness, especially with ischemic heart disease, stroke, cancer, chronic lung disease, arthritis, Alzheimer's disease, and Parkinson's disease.

Other risk factors associated with late-onset depression have been identified and may be used to identify subjects who will benefit from the present invention. Persistent insomnia, occurring in 5 to 10 percent of older adults, is a known risk factor for the subsequent onset of new cases of major depression both in middle-aged and older persons. Grief following the death of a spouse, relative, or close acquaintance (discussed below) also is an important risk factor for both major and minor depression. A final pathway to late-onset depression, suggested by computed tomography and magnetic resonance imaging studies, may involve structural, neuroanatomic factors. Enlarged lateral ventricles, cortical atrophy, increased white matter hyperintensities, decreased caudate size, and vascular lesions in the caudate nucleus appear to be especially prominent in late-onset depression associated with vascular risk factors. These findings have generated the vascular hypothesis of late-onset depression; namely, that even in the absence of a clear stroke, disorders that cause vascular damage, such as hypertension, coronary artery disease, and diabetes mellitus, may induce cerebral pathology that constitutes a vulnerability for depression.

Loss of a spouse, relative, or close acquaintance is common in late life. Bereavement is a natural response to such death. Its features, almost universally recognized, include crying and sorrow, anxiety and agitation, insomnia, and loss of appetite. This constellation of symptoms, while overlapping somewhat with major depression, does not by itself constitute a mental disorder. On the other hand, bereavement is an important and well-established risk factor for depression. At least 10 to 20 percent of widows and widowers develop clinically significant depression during the first year of bereavement. Only when symptoms persist for 2 months and longer after the loss does the DSM-IV permit a diagnosis of either adjustment disorder or major depressive disorder. Even though bereavement of less than 2 months' duration is not considered a mental disorder, it still warrants clinical attention (DSM-IV). The justification for clinical attention is that bereavement, as a highly stressful event, increases the probability of, and may cause or exacerbate, mental and somatic disorders. Without treatment, such late-onset depressions tend to persist, become chronic, and lead to further disability and impairments in general health, including alterations in endocrine and immune function.

Bereavement-associated depression often coexists with another type of emotional distress, which has been termed traumatic grief. The symptoms of traumatic grief, although not formalized as a mental disorder in DSM-IV, appear to be a mixture of symptoms of both pathological grief and post-traumatic stress disorder. Such symptoms are extremely disabling, associated with functional and health impairment and with persistent suicidal thoughts, and may well respond to pharmacotherapy. Increased illness and mortality from suicide are the most serious consequences of late-life depression.

The present invention provides a method of treating mood disorders of the types discussed above. Diagnosis of these disorders, or the identification of a patient at risk of one or more of them, is to be made by a physician or psychiatrist. It is presently believed that administration of an effective dose of an anti-Aβ agent results in the alleviation of the effects of the disorder from which the subject suffers, or even the elimination of the disorder completely. Diagnosis of depression in Alzheimer's patients may be more challenging than in other elderly patients. Recent diagnostic criteria for depression in Alzheimer's disease will aid the diagnosis [Olin, et al., Am. J Geriatr. Psychiatry 10:125-128 and 129-141 (2002)].

Major Depressive Episode may be diagnosed according to the following criteria: A) five (or more) of the following symptoms have been present during the same 2-week period and represent a change from previous functioning; at least one of the symptoms is either (1) depressed mood or (2) loss of interest or pleasure (excluding symptoms that are clearly due to a general medical condition, or mood-incongruent delusions or hallucinations): 1. depressed mood most of the day, nearly every day, as indicated by either subjective report (e.g., feels sad or empty) or observation made by others (e.g., appears tearful); 2. markedly diminished interest or pleasure in all, or almost all, activities most of the day, nearly every day (as indicated by either subjective account or observation made by others); 3. significant weight loss when not dieting or weight gain (e.g., a change of more than 5% of body weight in a month), or decrease or increase in appetite nearly every day; 4. insomnia or hypersomnia nearly every day; 5. psychomotor agitation or retardation nearly every day (observable by others, not merely subjective feelings of restlessness or being slowed down); 6. fatigue or loss of energy nearly every day; 7. feelings of worthlessness or excessive or inappropriate guilt (which may be delusional) nearly every day (not merely self-reproach or guilt about being sick); 8. diminished ability to think or concentrate, or indecisiveness, nearly every day (either by subjective account or as observed by others); 9. recurrent thoughts of death (not just fear of dying), recurrent suicidal ideation without a specific plan, or a suicide attempt or a specific plan for committing suicide; B) the symptoms do not meet criteria for a Mixed Episode; C) the symptoms cause clinically significant distress or impairment in social, occupational, or other important areas of functioning; D) the symptoms are not due to the direct physiological effects of a substance (e.g., a drug of abuse, a medication) or a general medical condition (e.g., hypothyroidism); and E) the symptoms are not better accounted for by Bereavement, i.e., after the loss of a loved one, the symptoms persist for longer than 2 months or are characterized by marked functional impairment, morbid preoccupation with worthlessness, suicidal ideation, psychotic symptoms, or psychomotor retardation.

Major Depressive Disorder (DSM-IV 296.3x) may be diagnosed according to the following criteria: A) at least one of the following abnormal moods which significantly interfered with the person's life—1. abnormal depressed mood most of the day, nearly every day, for at least 2 weeks or 2. abnormal loss of all interest and pleasure most of the day, nearly every day, for at least 2 weeks; B) at least five of the following symptoms have been present during the same 2 week depressed period: 1. abnormal depressed mood [as defined in criterion A]; 2. abnormal loss of all interest and pleasure [as defined in criterion A2]; 3. abnormal weight gain or loss (when not dieting) or increase/decrease in appetite; 4. sleep disturbance, either abnormal insomnia or abnormal hypersomnia; 5. activity disturbance, either abnormal agitation or abnormal slowing (observable by others); 6. abnormal fatigue or loss of energy; 7. abnormal self-reproach or inappropriate guilt; 8. abnormal poor concentration or indecisiveness; 9. abnormal morbid thoughts of death (not just fear of dying) or suicide; C) the symptoms are not due to a mood-incongruent psychosis; D) there has never been a Manic Episode, a Mixed Episode, or a Hypomanic Episode; E) the symptoms are not due to physical illness, alcohol, medication, or street drugs; and F) the symptoms are not due to normal bereavement.

Dysthymic disorder (DSM-IV 300.4) may be diagnosed according to the following criteria: A) depressed mood for most of the day, for more days than not, as indicated either by subjective account or observation by others, for at least 2 years; B) presence, while depressed, of two (or more) of: poor appetite or overeating, insomnia or hypersomnia, low energy or fatigue, low self-esteem, poor concentration or difficulty making decisions, and feelings of hopelessness; C) during the 2-year period of the disturbance, the person has never been without the symptoms in Criteria A and B for more than 2 months at a time; D) no Major Depressive Episode has been present during the first 2 years of the disturbance; i.e., the disturbance is not better accounted for by chronic Major Depressive Disorder or Major Depressive Disorder in partial remission (there may have been a previous Major Depressive Episode provided there was a full remission marked by there being no significant signs or symptoms for 2 months before development of the Dysthymic Disorder. In addition, after the initial 2 years of Dysthymic Disorder, there may be superimposed episodes of Major Depressive Disorder, in which case both diagnoses may be given when the criteria are met for a Major Depressive Episode.); E) there has never been a Manic Episode, a Mixed Episode, or a Hypomanic Episode, and criteria have never been met for Cyclothymic Disorder; F) the disturbance does not occur exclusively during the course of a chronic Psychotic Disorder, such as Schizophrenia or Delusional Disorder; G) the symptoms are not due to the direct physiological effects of a substance (e.g., a drug of abuse, a medication) or a general medical condition (e.g., hypothyroidism); and H) the symptoms cause clinically significant distress or impairment in social, occupational, or other important areas of functioning.

“Elderly subject” means a subject older than the average age of menopause for the species and culture (if relevant) of which the subject is a member, assuming adequate nutrition and general health. The average age of menopause for humans in the United States is 51 years of age [The Endocrine Society, 4350 East West Highway, Suite 500, Bethesda, Md. 20814-4426; www.endo-society.org]. The term “subject” or “patient” for purposes of the present invention is any warm-blooded animal such as, but not limited to, a mouse, guinea pig, dog, horse, or human. Preferably, the subject is a mammal, more preferably rodent or primate, and most preferably, human.

“Administering” is the act of introducing a substance into the body of a subject, and may be achieved by oral, intravenous, intraperitoneal, subcutaneous, intramuscular, or intraparenchimal routes, among others. The antibodies are administered to a subject as identified herein using standard administration techniques, such as by intravenous, intraperitoneal, subcutaneous, pulmonary, transdermal, intramuscular, intranasal, buccal, sublingual, infusion, or suppository administration. The preferred routes of administration are intravenous, subcutaneous, and intraperitoneal. More preferred is either intravenous or subcutaneous.

“Effective dose” means an amount of a substance that leads to measurable and beneficial effects, i.e. significant efficacy. The particular effective amount or dose of compound administered according to this invention will of course be determined by the particular circumstances surrounding the case, including the compound administered, the route of administration, the particular condition being treated, and similar considerations. A typical daily dose will contain from about 0.01 mg/kg to about 100 mg/kg of the active compound of this invention. Preferably, daily doses will be about 0.05 mg/kg to about 50 mg/kg, more preferably from about 0.1 mg/kg to about 25 mg/kg. The frequency of dosing may be daily or once, twice, three times or more per week or per month, as needed to effectively treat the condition.

“Anti-Aβ antibody” means an immunoglobulin molecule (preferably an IgG) that recognizes, binds, and (or) sequesters Aβ peptide.

“Aβ peptide” and “Aβ ” refer to a peptide that is derived from amyloid precursor protein (Alzheimer's disease amyloid A4 protein [Precursor], “APP”) by proteolytic cleavage. Full-length Aβ peptides are from 39 to 43 amino acids long in humans, for example. Full length Aβ peptide may undergo further cleavage in vivo to produce Aβ fragments that are shorter at the N-terminus, at the C-terminus, or both, by one to several amino acids. Full-length Aβ peptide or fragments thereof may be used also as antigens to raise antibodies that bind Aβ peptide. Among the many Aβ peptide fragments used for this purpose, the Aβ13-28 fragment (conjugated via m-maleimidobenzoyl-N-hydroxysuccinimide ester to an anti-CD3 antibody) was used to raise antibody 266 [Seubert, P. et al., Nature 359:325-327.(1992)].

“Conditions associated with Aβ” include clinical or pre-clinical Alzheimer's disease, Down's syndrome, and chronic amyloid angiopathy [Grabowski T. J., et al., Ann. Neurol. 49: 697-705 (2001); Vinters H. V., Ann. Neurol. 49: 691-3 (2001)]. Regardless of the cause, the diagnosis of AD is made clinically by the finding of progressive memory loss with increasing inability to participate in activities of daily living. Mild cognitive impairment may be associated with pre-clinical Alzheimer's disease.

By “antibody” is meant a whole antibody, including without limitation an animal-derived antibody (e.g., murine), chimeric, humanized, human sequence, recombinant, transgenic, grafted and single chain antibody, and the like, or any fusion proteins, conjugates, fragments, or derivatives thereof An antibody comprises protein resembling an antibody in the broadest sense in that the protein comprises a binding site for an antigen, which binding site is comprised of three pairs of complementarity determining regions. Antibody includes a whole immunoglobulin molecule, a monoclonal antibody, a chimeric antibody, a humanized antibody; a human antibody, or an immunologically effective fragment of any of these. An antibody fragment, or simply fragment, means an Fv, a disulfide linked Fv, scFv, Fab, Fab′, or F(ab′)₂ fragment, which terms are well known in the art. In some contexts, herein, fragments will be mentioned specifically for emphasis; nevertheless, it will be understood that regardless of whether fragments are specified, the term “antibody” includes such fragments as well as single-chain forms. As long as a protein retains the ability specifically to bind its intended target, it is included within the term “antibody.” Also included within the definition “antibody” are single chain forms. Preferably, but not necessarily, the antibodies useful in the invention are produced recombinantly. Antibodies may or may not be glycosylated, though glycosylated antibodies are preferred under some circumstances, such as when prolonged residence in the body is desirable, or when minimum risk of developing neutralizing antibodies. Antibodies, except perhaps for certain types in which cross-linking between chains is accomplished by peptide or other chemical chains, are properly cross-linked via disulfide bonds.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. IgG isotypes are preferred. Of the IgG subclasses, IgG1 and IgG4 are preferred.

By “humanized antibody” is meant an antibody that is composed partially or fully of amino acid sequences derived from a human antibody germline by altering the sequence of an antibody having non-human complementarity determining regions (CDR). A humanized immunoglobulin does not encompass a chimeric antibody, having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms.

Preparation of antibodies for use in the present invention may be carried out by methods well known in the art, including preparing monoclonal antibodies using well known techniques and screening for high affinity antibodies, or by first identifying a monoclonal antibody having reasonably high affinity and then improving the affinity using well known methods [e.g., U.S. Pat. Nos. 5,976,562, 5,824514, 5,817,483, 5,814,476, 5,763,192, 5,723,323; WO97/29131; Huse, W. D., et al., Internat'l Rev. Immunol. 10:129-137 (1993); Yelton, D. E., et al., J. Immunol. 155:1994-2004 (1995); Wu, H., et al., Proc. Natl. Acad. Sci. (USA) 95:6037-6042 (1998); Crameri, A., et al., Nature Medicine 2:100-103 (1996); Stemmer, Proc. Natl. Acad. Sci. (USA) 91:10747-10751 (1994); Stemmer, Nature 370:389-391 (1994), the portion of each of which having to do with preparation of antibodies is incorporated herein by reference].

The antibodies used in the present invention will most advantageously be expressed in recombinant hosts and purified using well known techniques [Page, M. J. & Sydenham, M. A., Bio/Technol. 9, 64-68 (1991); Carroll, A. R., et al., Mol. ininunol. 29, 821-827 (1992); Coloma, M. J., et al., J. Immunol. Meth. 152, 89-104 (1992); Bebbington, C. R., et al., Bio/Technol. 10, 169-175 (1992); Deyev, S., et al., FEBS Lett. 330, 111-113 (1993); Bender, E., et al., Hum. Antibodies Hybridomas 4, 74-79 (1993); Norderhaug, L., et al., J. Immunol. Meth. 204, 77-87 (1997); Poul, M. A., et al., Eur. J. Immunol. 25, 2005-2009 (1995), each of which is incorporated herein by reference].

A preferred antibody for use in the present invention is 266, a humanized form of 266, an antibody that binds to the same epitope on Aβ that 266 binds, any antibody comprised of the CDRs of 266, and any antibody that competitively inhibits the binding of 266 and human Aβ. The skilled reader will know how to determine, from among many possible methods that are well known, whether any particular antibody competitively inhibits the binding of 266 and human Aβ. For example, a comparative ELISA method could be used. Wells of a 96-well ELISA plate (e.g., Nunc-Immuno plate, Cat #439454, NalgeNunc) are coated with Aβ peptide (1-42 is convenient, but other lengths could be used also), optionally conjugated to a larger protein such as albumin. After washing the wells, they are blocked as appropriate, and then rinsed and dried appropriately. A mixture of biotinylated 266 antibody (e.g., mouse or humanized; at 0.3 μg/ml final concentration, for example) and a competitor antibody (starting at 750 μg/ml final concentration and serial 3-fold dilutions) are added in a final volume of 100 μl per well. No-competitor and background controls are run. The ELISA plate is incubated at an appropriate temperature for an appropriate length of time, and then the wells are washed. After washing the wells, HRP-conjugated streptavidin (Cat #21124, Pierce), or equivalent, is added to each well (e.g., 100 μl of 1 μg/ml). The plate is incubated at room temperature for 30 min and washed. For color development, 100 μl/well of ABTS Peroxidase Substrate (Kirkegaard & Perry Laboratories), or equivalent, is added. Color development is stopped and absorbance is read (e.g., at 415 nm). The absorbances are plotted against the log of the competitor concentration, curves are fitted to the data points (e.g., using Prism or equivalent) and the IC50 determined using methods well known in the art. An antibody having an IC50 within about 100-fold of that of 266 is considered to competitively inhibit its binding.

Antibody 266 has the following amino acid sequences as CDRs:

| | |
|---|---|
| light chain CDR1: | |
| 1 5 | |
| Arg Ser Ser Gln Ser Leu Ile Tyr Ser | (SEQ ID NO: 1) |
| 10 15 | |
| Asp Gly Asn Ala Tyr Leu His | |
| light chain CDR2: | |
| 1 5 | |
| Lys Val Ser Asn Arg Phe Ser | (SEQ ID NO: 2) |
| light chain CDR3: | |
| 1 5 | |
| Ser Gln Ser Thr His Val Pro Trp Thr | (SEQ ID NO: 3) |
| heavy chain CDR1: | |
| 1 5 | |
| Arg Tyr Ser Met Ser | (SEQ ID NO: 4) |
| heavy chain CDR2: | |
| 1 5 | |
| Gln Ile Asn Ser Val Gly Asn Ser Thr | (SEQ ID NO: 5) |
| 10 15 | |
| Tyr Tyr Pro Asp Thr Val Lys Gly | |
| and, | |
| heavy chain CDR3: | |
| 1 | |
| Gly Asp Tyr. | (SEQ ID NO: 6) |

In humanized versions of 266, human framework regions may optionally have substitutions of one to several residues from mouse 266 for the purpose of maintaining the strength or specificity of the binding of humanized antibody 266 [see, Holtzman, et al., WO00/ 62801]. A preferred light chain variable region of a humanized 266 antibody for use in the present invention has the following amino acid sequence:

| | |
|---|---|
| 1 5 10 15 | |
| Asp Xaa Val Met Thr Gln Xaa Pro Leu Ser Leu Pro Val Xaa Xaa Gly | (SEQ ID NO: 7) |
| 20 25 30 | |
| Gln Pro Ala Ser Ile Ser Cys Arg Ser Ser Gln Ser Leu Xaa Tyr Ser | |
| 35 40 45 | |
| Asp Gly Asn Ala Tyr Leu His Trp Phe Leu Gln Lys Pro Gly Gln Ser | |
| 50 55 60 | |
| Pro Xaa Leu Leu Ile Tyr Lys Val Ser Asn Arg Phe Ser Gly Val Pro | |
| 65 70 75 80 | |
| Asp Arg Phe Ser Gly Ser Gly Ser Gly Thr Asp Phe Thr Leu Lys Ile | |
| 85 90 95 | |
| Ser Arg Val Glu Ala Glu Asp Xaa Gly Val Tyr Tyr Cys Ser Gln Ser | |
| 100 105 110 | |
| Thr His Val Pro Trp Thr Phe Gly Xaa Gly Thr Xaa Xaa Glu Ile Lys | |
| Arg | |

wherein:
Xaa at position 2 is Val or Ile;
Xaa at position 7 is Ser or Thr;
Xaa at position 14 is Thr or Ser;
Xaa at position 15 is Leu or Pro;
Xaa at position 30 is Ile or Val;
Xaa at position 50 is Arg, Gln, or Lys;
Xaa at position 88 is Val or Leu;
Xaa at position 105 is Gln or Gly;
Xaa at position 108 is Lys or Arg; and
Xaa at position 109 is Val or Leu.

A preferred heavy chain variable region of a humanized 266 antibody for use in the present invention has the following amino acid sequence:

| | |
|---|---|
| 1 5 10 15 | |
| Xaa Val Gln Leu Val Glu Xaa Gly Gly Gly Leu Val Gln Pro Gly Gly | (SEQ ID NO: 8) |
| 20 25 30 | |
| Ser Leu Arg Leu Ser Cys Ala Ala Ser Gly Phe Thr Phe Ser Arg Tyr | |
| 35 40 45 | |
| Ser Met Ser Trp Val Arg Gln Ala Pro Gly Lys Gly Leu Xaa Leu Val | |
| 50 55 60 | |
| Ala Gln Ile Asn Ser Val Gly Asn Ser Thr Tyr Tyr Pro Asp Xaa Val | |
| 65 70 75 80 | |
| Lys Gly Arg Phe Thr Ile Ser Arg Asp Asn Xaa Xaa Asn Thr Leu Tyr | |
| 85 90 95 | |
| Leu Gln Met Asn Ser Leu Arg Ala Xaa Asp Thr Ala Val Tyr Tyr Cys | |
| 100 105 110 | |
| Ala Ser Gly Asp Tyr Trp Gly Gln Gly Thr Xaa Val Thr Val Ser Ser | |

wherein:
Xaa at position 1 is Glu or Gln;
Xaa at position 7 is Ser or Leu;
Xaa at position 46 is Glu, Val, Asp, or Ser;
Xaa at position 63 is Thr or Ser;
Xaa at position 75 is Ala, Ser, Val, or Thr;
Xaa at position 76 is Lys or Arg;
Xaa at position 89 is Glu or Asp; and
Xaa at position 107 is Leu or Thr.

A particularly preferred light chain variable region of a humanized 266 antibody for use in the present invention has the following amino acid sequence:

| | |
|---|---|
| 1 5 10 15 | |
| Asp Val Val Met Thr Gln Ser Pro Leu Ser Leu Pro Val Thr Leu Gly | (SEQ ID NO: 9) |
| 20 25 30 | |
| Gln Pro Ala Ser Ile Ser Cys Arg Ser Ser Gln Ser Leu Ile Tyr Ser | |
| 35 40 45 | |
| Asp Gly Asn Ala Tyr Leu His Trp Phe Leu Gln Lys Pro Gly Gln Ser | |
| 50 55 60 | |
| Pro Arg Leu Leu Ile Tyr Lys Val Ser Asn Arg Phe Ser Gly Val Pro | |
| 65 70 75 80 | |
| Asp Arg Phe Ser Gly Ser Gly Ser Gly Thr Asp Phe Thr Leu Lys Ile | |
| 85 90 95 | |
| Ser Arg Val Glu Ala Glu Asp Val Gly Val Tyr Tyr Cys Ser Gln Ser | |
| 100 105 110 | |
| Thr His Val Pro Trp Thr Phe Gly Gln Gly Thr Lys Val Glu Ile Lys | |
| Arg. | |

A particularly preferred heavy chain variable region of a humanized 266 antibody for use in the present invention has the following amino acid sequence:

| | |
|---|---|
| 1 5 10 15 | |
| Glu Val Gln Leu Val Glu Ser Gly Gly Gly Leu Val Gln Pro Gly Gly | (SEQ ID NO: 10) |
| 20 25 30 | |
| Ser Leu Arg Leu Ser Cys Ala Ala Ser Gly Phe Thr Phe Ser Arg Tyr | |
| 35 40 45 | |
| Ser Met Ser Trp Val Arg Gln Ala Pro Gly Lys Gly Leu Glu Leu Val | |
| 50 55 60 | |
| Ala Gln Ile Asn Ser Val Gly Asn Ser Thr Tyr Tyr Pro Asp Thr Val | |
| 65 70 75 80 | |
| Lys Gly Arg Phe Thr Ile Ser Arg Asp Asn Ala Lys Asn Thr Leu Tyr | |
| 85 90 95 | |
| Leu Gln Met Asn Ser Leu Arg Ala Glu Asp Thr Ala Val Tyr Tyr Cys | |
| 100 105 110 | |
| Ala Ser Gly Asp Tyr Trp Gly Gln Gly Thr Leu Val Thr Val Ser Ser. | |

A preferred light chain for a humanized 266 antibody for use in the present invention has the amino acid sequence:

| | |
|---|---|
| 1 5 10 15 | |
| Asp Val Val Met Thr Gln Ser Pro Leu Ser Leu Pro Val Thr Leu | (SEQ ID NO: 11) |
| 20 25 30 | |
| Gly Gln Pro Ala Ser Ile Ser Cys Arg Ser Ser Gln Ser Leu Ile | |
| 35 40 45 | |
| Tyr Ser Asp Gly Asn Ala Tyr Leu His Trp Phe Leu Gln Lys Pro | |
| 50 55 60 | |
| Gly Gln Ser Pro Arg Leu Leu Ile Tyr Lys Val Ser Asn Arg Phe | |
| 65 70 75 | |
| Ser Gly Val Pro Asp Arg Phe Ser Gly Ser Gly Ser Gly Thr Asp | |
| 80 85 90 | |
| Phe Thr Leu Lys Ile Ser Arg Val Glu Ala Glu Asp Val Gly Val | |
| 95 100 105 | |
| Tyr Tyr Cys Ser Gln Ser Thr His Val Pro Trp Thr Phe Gly Gln | |
| 110 115 120 | |
| Gly Thr Lys Val Glu Ile Lys Arg Thr Val Ala Ala Pro Ser Val | |
| 125 130 135 | |
| Phe Ile Phe Pro Pro Ser Asp Glu Gln Leu Lys Ser Gly Thr Ala | |
| 140 145 150 | |
| Ser Val Val Cys Leu Leu Asn Asn Phe Tyr Pro Arg Glu Ala Lys | |
| 155 160 165 | |
| Val Gln Trp Lys Val Asp Asn Ala Leu Gln Ser Gly Asn Ser Gln | |
| 170 175 180 | |
| Glu Ser Val Thr Glu Gln Asp Ser Lys Asp Ser Thr Tyr Ser Leu | |
| 185 190 195 | |
| Ser Ser Thr Leu Thr Leu Ser Lys Ala Asp Tyr Glu Lys His Lys | |
| 200 205 210 | |
| Val Tyr Ala Cys Glu Val Thr His Gln Gly Leu Ser Ser Pro Val | |
| 215 | |
| Thr Lys Ser Phe Asn Arg Gly Glu Cys. | |

A preferred heavy chain for a humanized 266 antibody for use in the present invention has the amino acid,sequence:

| | |
|---|---|
| 1 5 10 15 | |
| Glu Val Gln Leu Val Glu Ser Gly Gly Gly Leu Val Gln Pro Gly | (SEQ ID NO: 12) |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Glu Leu Val Ala Gln Ile Asn Ser Val Gly Asn Ser Thr Tyr Tyr | |
| 65 70 75 | |
| Pro Asp Thr Val Lys Gly Arg Phe Thr Ile Ser Arg Asp Asn Ala | |
| 80 85 90 | |
| Lys Asn Thr Leu Tyr Leu Gln Met Asn Ser Leu Arg Ala Glu Asp | |
| 95 100 105 | |
| Thr Ala Val Tyr Tyr Cys Ala Ser Gly Asp Tyr Trp Gly Gln Gly | |
| 110 115 120 | |
| Thr Leu Val Thr Val Ser Ser Ala Ser Thr Lys Gly Pro Ser Val | |
| 125 130 135 | |
| Phe Pro Leu Ala Pro Ser Ser Lys Ser Thr Ser Gly Gly Thr Ala | |
| 140 145 150 | |
| Ala Leu Gly Cys Leu Val Lys Asp Tyr Phe Pro Glu Pro Val Thr | |
| 155 160 165 | |
| Val Ser Trp Asn Ser Gly Ala Leu Thr Ser Gly Val His Thr Phe | |
| 170 175 180 | |
| Pro Ala Val Leu Gln Ser Ser Gly Leu Tyr Ser Leu Ser Ser Val | |
| 185 190 195 | |
| Val Thr Val Pro Ser Ser Ser Leu Gly Thr Gln Thr Tyr Ile Cys | |
| 200 205 210 | |
| Asn Val Asn His Lys Pro Ser Asn Thr Lys Val Asp Lys Lys Val | |
| 215 220 225 | |
| Glu Pro Lys Ser Cys Asp Lys Thr His Thr Cys Pro Pro Cys Pro | |
| 230 235 240 | |
| Ala Pro Glu Leu Leu Gly Gly Pro Ser Val Phe Leu Phe Pro Pro | |
| 245 250 255 | |
| Lys Pro Lys Asp Thr Leu Met Ile Ser Arg Thr Pro Glu Val Thr | |
| 260 265 270 | |
| Cys Val Val Val Asp Val Ser His Glu Asp Pro Glu Val Lys Phe | |
| 275 280 285 | |
| Asn Trp Tyr Val Asp Gly Val Glu Val His Asn Ala Lys Thr Lys | |
| 290 295 300 | |
| Pro Arg Glu Glu Gln Tyr Asn Ser Thr Tyr Arg Val Val Ser Val | |
| 305 310 315 | |
| Leu Thr Val Leu His Gln Asp Trp Leu Asn Gly Lys Glu Tyr Lys | |
| 320 325 330 | |
| Cys Lys Val Ser Asn Lys Ala Leu Pro Ala Pro Ile Glu Lys Thr | |
| 335 340 345 | |
| Ile Ser Lys Ala Lys Gly Gln Pro Arg Glu Pro Gln Val Tyr Thr | |
| 350 355 360 | |
| Leu Pro Pro Ser Arg Asp Glu Leu Thr Lys Asn Gln Val Ser Leu | |
| 365 370 375 | |
| Thr Cys Leu Val Lys Gly Phe Tyr Pro Ser Asp Ile Ala Val Glu | |
| 380 385 390 | |
| Trp Glu Ser Asn Gly Gln Pro Glu Asn Asn Tyr Lys Thr Thr Pro | |
| 395 400 405 | |
| Pro Val Leu Asp Ser Asp Gly Ser Phe Phe Leu Tyr Ser Lys Leu | |
| 410 415 420 | |
| Thr Val Asp Lys Ser Arg Trp Gln Gln Gly Asn Val Phe Ser Cys | |
| 425 430 435 | |
| Ser Val Met His Glu Ala Leu His Asn His Tyr Thr Gln Lys Ser | |
| 440 | |
| Leu Ser Leu Ser Pro Gly Lys. | |

Another preferred antibody for use in the present invention is an analog of 266, in which an N-glycosylation site within CDR2 of the heavy chain (SEQ ID NO:5) is engineered so as not to be glycosylated. Such an analog comprises a light chain and a heavy chain, wherein the light chain comprises the three light chain complementarity determining regions (CDRS) from mouse monoclonal antibody 266 (SEQ ID NO: 1-3), and wherein the heavy chain comprises heavy chain CDR1 and CDR3 from mouse monoclonal antibody 266 (SEQ ID NO: 4 and 6, respectively), and a heavy chain CDR2 having the sequence given by SEQ ID NO:13:

| | |
|---|---|
| 1 5 | |
| Gln Ile Asn Ser Val Gly | (SEQ ID NO: 13) |
| 10 | |
| Xaa Xaa Xaa Tyr Tyr Pro | |
| 15 | |
| Asp Thr Val Lys Gly | |

wherein,
Xaa at position 7 is any amino acid, provided that if Xaa at position 8 is neither Asp nor Pro and Xaa at position 9 is Ser or Thr, then Xaa at position 7 is not Asn;
Xaa at position 8 is any amino acid, provided that if Xaa at position 7 is Asn and Xaa at position 9 is Ser or Thr, then Xaa at position 8 is Asp or Pro; and
Xaa at position 9 is any amino acid, provided that if Xaa at position 7 is Asn and Xaa at position 8 is neither Asp nor Pro, then Xaa at position 9 is neither Ser nor Thr.

By “any amino acid” is meant any naturally occurring amino acid. Preferred naturally-occurring amino acids are Ala, Cys, Asp, Glu, Phe, Gly, His, Ile, Lys, Leu, Met, Asn, Pro, Gln, Arg, Ser, Thr, Val, Trp, and Tyr.

A preferred group of antibodies are those having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is SEQ ID NO:13, wherein:
Xaa at position 7 of SEQ ID NO: 13 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 8 is neither Asp nor Pro and Xaa at position 9 is Ser or Thr, then Xaa at position 7 is not Asn;
Xaa at position 8 of SEQ ID NO: 13 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 7 is Asn and Xaa at position 9 is Ser or Thr, then Xaa at position 8 is Asp or Pro; and
Xaa at position 9 of SEQ ID NO:13.is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 7 is Asn and Xaa at position 8 is neither Asp nor Pro, then Xaa at position 9 is neither Ser nor Thr.

Another description of the preferred group is: antibodies or fragments thereof having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

| | |
|---|---|
| 1 5 | |
| Gln Ile Asn Ser Val Gly | (SEQ ID NO: 14) |
| 10 | |
| Xaa Xaa Xaa Tyr Tyr Pro | |
| 15 | |
| Asp Thr Val Lys Gly | |

wherein:
Xaa at position 7 of SEQ ID NO:14 is selected from the group consisting of Ala, Cys, Asp, Glu, Phe, Gly, His, Ile, Lys, Leu, Met, Pro, Gln, Arg, Ser, Thr, Val, Trp, and Tyr;
Xaa at position 8 of SEQ ID NO:14 is selected from the group consisting of Ala, Cys, Asp, Glu, Phe, Gly, His, Ile, Lys, Leu, Met, Asn, Pro, Gln, Arg, Ser, Thr, Val, Trp, and Tyr; and

Xaa at position 9 of SEQ ID NO:14 is selected from the group consisting of Ala, Cys, Asp, Glu, Phe, Gly, His, Ile, Lys, Leu, Met, Asn, Pro, Gln, Arg, Ser, Thr, Val, Trp, and Tyr;

| | |
|---|---|
| 1 5 | |
| Gln Ile Asn Ser Val Gly | (SEQ ID NO: 15) |
| 10 | |
| Xaa Xaa Xaa Tyr Tyr Pro | |
| Asp Thr Val Lys Gly | |

wherein:
Xaa at position 7 of SEQ ID NO: 15 is Asn;
Xaa at position 8 of SEQ ID NO:15 is selected from the group consisting of Ala, Cys, Asp, Glu, Phe, Gly, His, Ile, Lys, Leu, Met, Asn, Pro, Gln, Arg, Ser, Thr, Val, Trp, and Tyr; and

Xaa at position 9 of SEQ ID NO:15 is selected from the group consisting of Ala, Cys, Asp, Glu, Phe, Gly, His, Ile, Lys, Leu, Met, Asn, Pro, Gln, Arg, Val, Trp, and Tyr; and

(SEQ ID NO: 16)

1 5
Gln Ile Asn Ser Val Gly Xaa Xaa Xaa
10 15
Tyr Tyr Pro Asp Thr Val Lys Gly

wherein:
Xaa at position 7 of SEQ ID NO: 16 is Asn;
Xaa at position 8 of SEQ ID NO: 16 is selected from the group consisting of Asp and Pro; and
Xaa at position 9 of SEQ ID NO: 16 is selected from the group consisting of Ser and Thr.

Preferred sequences for CDR2 of the heavy chain include those in which only a single amino acid is changed, those in which only two amino acids are changed, or all three are changed. It is preferred to replace Asn at position 7, or to replace Thr at position 9, or to replace both. Conservative substitutions at one, two, or all three positions are preferred. The most preferred species are those in which Asn at position 7 is replaced with Ser or Thr.

Preferred deglycosylated 266 antibodies for use in the present invention are those in which in CDR2 of the heavy chain (i.e., within SEQ ID NO: 13, as described above):
Xaa at position 7 is selected from the group consisting of Ala, Gly, His, Asn, Gln, Ser, and Thr, provided that if Xaa at position 9 is Ser or Thr, then Xaa at position 7 is not Asn;
Xaa at position 8 is selected from the group consisting of Ala, Gly, His, Asn, Gln, Ser, and Thr; and
Xaa at position 9 is selected from the group consisting of Ala, Gly, His, Asn, Gln, Ser, and Thr, provided that if Xaa at position 7 is Asn, then Xaa at position 9 is neither Ser nor Thr.

An alternate description of preferred declycogsylated 266 antibodies is: antibodies or fragments thereof having as light chain CDRI-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

(SEQ ID NO: 17)

1 5
Gln Ile Asn Ser Val Gly Xaa Xaa Xaa
10 15
Tyr Tyr Pro Asp Thr Val Lys Gly

wherein:
Xaa at position 7 of SEQ ID NO: 17 is selected from the group consisting of Ala, Gly, His, Gln, Ser, and Thr;
Xaa at position 8 of SEQ ID NO: 17 is selected from the group consisting of Ala, Gly, His, Asn, Gln, Ser, and Thr; and

Xaa at position 9 of SEQ ID NO: 17 is selected from the group consisting of Ala, Gly, His, Asn, Gln, Ser, and Thr; and

(SEQ ID NO: 18)

1 5
Gln Ile Asn Ser Val Gly Xaa Xaa Xaa
10 15
Tyr Tyr Pro Asp Thr Val Lys Gly

wherein:
Xaa at position 7 of SEQ ID NO: 18 is Asn;
Xaa at position 8 of SEQ ID NO:1 8 is selected from the group consisting of Ala, Gly, His, Asn, Gln, Ser, and Thr; and
Xaa at position 9 of SEQ ID NO: 18 is selected from the group consisting of Ala, Gly, His, Asn, and Gln.

A preferred humanized antibody for use in the present invention has the light chain variable region of SEQ ID NO:7 and a heavy chain variable region of SEQ ID NO:19

| | |
|---|---|
| 1 5 10 15 | |
| Xaa Val Gln Leu Val Glu Xaa Gly Gly Gly Leu Val Gln Pro Gly | (SEQ ID NO: 19) |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Xaa Leu Val Ala Gln Ile Asn Ser Val Gly Xaa Xaa Xaa Tyr Tyr | |
| 65 70 75 | |
| Pro Asp Xaa Val Lys Gly Arg Phe Thr Ile Ser Arg Asp Asn Xaa | |
| 80 85 90 | |
| Xaa Asn Thr Leu Tyr Leu Gln Met Asn Ser Leu Arg Ala Xaa Asp | |
| 95 100 105 | |
| Thr Ala Val Tyr Tyr Cys Ala Ser Gly Asp Tyr Trp Gly Gln Gly | |
| 110 | |
| Thr Xaa Val Thr Val Ser Ser | |

wherein:
Xaa at position 1 is Glu or Gln;
Xaa at position 7 is Ser or Leu;
Xaa at position 46 is Glu, Val, Asp, or Ser;
Xaa at position 56 is any amino acid, provided that if Xaa at position 57 is neither Asp nor Pro and Xaa at position 59 is Ser or Thr, then Xaa at position 56 is not Asn;
Xaa at position 57 is any amino acid, provided that if Xaa at position 56 is Asn and Xaa at position 58 is Ser or Thr, then Xaa at position 57 is Asp or Pro; and
Xaa at position 58 is any amino acid, provided that if Xaa at position 56 is Asn and Xaa at position 57 is neither Asp nor Pro, then Xaa at position 58 is neither Ser nor Thr
Xaa at position 63 is Thr or Ser;
Xaa at position 75 is Ala, Ser, Val, or Thr;
Xaa at position 76 is Lys or Arg;
Xaa at position 89 is Glu or Asp; and
Xaa at position 107 is Leu or Thr.

A preferred humanized antibody for use in the present invention has the light chain variable region of SEQ ID NO:9 and a heavy chain variable region of SEQ ID NO:20:

| | |
|---|---|
| 1 5 10 15 | |
| Glu Val Gln Leu Val Glu Ser Gly Gly Gly Leu Val Gln Pro Gly | (SEQ ID NO: 20) |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Glu Leu Val Ala Gln Ile Asn Ser Val Gly Xaa Xaa Xaa Tyr Tyr | |
| 65 70 75 | |
| Pro Asp Thr Val Lys Gly Arg Phe Thr Ile Ser Arg Asp Asn Ala | |
| 80 85 90 | |
| Lys Asn Thr Leu Tyr Leu Gln Met Asn Ser Leu Arg Ala Glu Asp | |
| 95 100 105 | |
| Thr Ala Val Tyr Tyr Cys Ala Ser Gly Asp Tyr Trp Gly Gln Gly | |
| 110 | |
| Thr Leu Val Thr Val Ser Ser. | |

wherein:
Xaa at position 56 is any amino acid, provided that if Xaa at position 57 is neither Asp nor Pro and Xaa at position 59 is Ser or Thr, then Xaa at position 56 is not Asn;
Xaa at position 57 is any amino acid, provided that if Xaa at position 56 is Asn and Xaa at position 58 is Ser or Thr, then Xaa at position 57 is Asp or Pro; and
Xaa at position 58 is any amino acid, provided that if Xaa at position 56 is Asn and Xaa at position 57 is neither Asp nor Pro, then Xaa at position 58 is neither Ser nor Thr.

A preferred humanized antibody for use in the present invention has the light chain variable region of SEQ ID NO:1 1 and a heavy chain given by SEQ ID NO:21:

| | |
|---|---|
| 1 5 10 15 | |
| Glu Val Gln Leu Val Glu Ser Gly Gly Gly Leu Val Gln Pro Gly | (SEQ ID NO: 21) |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Glu Leu Val Ala Gln Ile Asn Ser Val Gly Xaa Xaa Xaa Tyr Tyr | |
| 65 70 75 | |
| Pro Asp Thr Val Lys Gly Arg Phe Thr Ile Ser Arg Asp Asn Ala | |
| 80 85 90 | |
| Lys Asn Thr Leu Tyr Leu Gln Met Asn Ser Leu Arg Ala Glu Asp | |
| 95 100 105 | |
| Thr Ala Val Tyr Tyr Cys Ala Ser Gly Asp Tyr Trp Gly Gln Gly | |
| 110 115 120 | |
| Thr Leu Val Thr Val Ser Ser Ala Ser Thr Lys Gly Pro Ser Val | |
| 125 130 135 | |
| Phe Pro Leu Ala Pro Ser Ser Lys Ser Thr Ser Gly Gly Thr Ala | |
| 140 145 150 | |
| Ala Leu Gly Cys Leu Val Lys Asp Tyr Phe Pro Glu Pro Val Thr | |
| 155 160 165 | |
| Val Ser Trp Asn Ser Gly Ala Leu Thr Ser Gly Val His Thr Phe | |
| 170 175 180 | |
| Pro Ala Val Leu Gln Ser Ser Gly Leu Tyr Ser Leu Ser Ser Val | |
| 185 190 195 | |
| Val Thr Val Pro Ser Ser Ser Leu Gly Thr Gln Thr Tyr Ile Cys | |
| 200 205 210 | |
| Asn Val Asn His Lys Pro Ser Asn Thr Lys Val Asp Lys Lys Val | |
| 215 220 225 | |
| Glu Pro Lys Ser Cys Asp Lys Thr His Thr Cys Pro Pro Cys Pro | |
| 230 235 240 | |
| Ala Pro Glu Leu Leu Gly Gly Pro Ser Val Phe Leu Phe Pro Pro | |
| 245 250 255 | |
| Lys Pro Lys Asp Thr Leu Met Ile Ser Arg Thr Pro Gln Val Thr | |
| 260 265 270 | |
| Cys Val Val Val Asp Val Ser His Gln Asp Pro Gln Val Lys Phe | |
| 275 280 285 | |
| Asn Trp Tyr Val Asp Gly Val Glu Val His Asn Ala Lys Thr Lys | |
| 290 295 300 | |
| Pro Arg Glu Gln Gln Tyr Asn Ser Thr Tyr Arg Val Val Ser Val | |
| 305 310 315 | |
| Leu Thr Val Leu His Gln Asp Trp Leu Asn Gly Lys Glu Tyr Lys | |
| 320 325 330 | |
| Cys Lys Val Ser Asn Lys Ala Leu Pro Ala Pro Ile Gln Lys Thr | |
| 335 340 345 | |
| Ile Ser Lys Ala Lys Gly Gln Pro Arg Gln Pro Gln Val Tyr Thr | |
| 350 355 360 | |
| Leu Pro Pro Ser Arg Asp Gln Leu Thr Lys Asn Gln Val Ser Leu | |
| 365 370 375 | |
| Thr Cys Leu Val Lys Gly Phe Tyr Pro Ser Asp Ile Ala Val Gln | |
| 380 385 390 | |
| Trp Gln Ser Asn Gly Gln Pro Gln Asn Asn Tyr Lys Thr Thr Pro | |
| 395 400 405 | |
| Pro Val Leu Asp Ser Asp Gly Ser Phe Phe Leu Tyr Ser Lys Leu | |
| 410 415 420 | |
| Thr Val Asp Lys Ser Arg Trp Gln Gln Gly Asn Val Phe Ser Cys | |
| 425 430 435 | |
| Ser Val Met His Gln Ala Leu His Asn His Tyr Thr Gln Lys Ser | |
| 440 | |
| Leu Ser Leu Ser Pro Gly Lys | |

wherein:
Xaa at position 56 is any amino acid, provided that if Xaa at position 57 is neither Asp nor Pro and Xaa at position 59 is Ser or Thr, then Xaa at position 56 is not Asn;
Xaa at position 57 is any amino acid, provided that if Xaa at position 56 is Asn and Xaa at position 58 is Ser or Thr, then Xaa at position 57 is Asp or Pro; and
Xaa at position 58 is any amino acid, provided that if Xaa at position 56 is Asn and Xaa at position 57 is neither Asp nor Pro, then Xaa at position 58 is neither Ser nor Thr.

Preferred deglycosylated 266 antibodies having the heavy variable region according to SEQ ID NO:19, SEQ ID NO:20, and SEQ ID NO:21 are those wherein:
Xaa at position 56 is selected from the group consisting of Ala, Gly, His, Asn, Gln, Ser, and Thr, provided that if Xaa at position 58 is Ser or Thr, then Xaa at position 56 is not Asn;
Xaa at position 57 is selected from the group consisting of Ala, Gly, His, Asn, Gin, Ser, and Thr; and
Xaa at position 58 is selected from the group consisting of Ala, Gly, His, Asn, Gln, Ser, and Thr, provided that if Xaa at position 56 is Asn, then Xaa at position 58 is neither Ser nor Thr.

Preferred sequences for CDR2 (positions 56, 57, and 58) of the heavy chain SEQ ID NO: 19, SEQ ID NO:20, and SEQ ID NO:21 include those in which only a single amino acid is changed, those in which only two amino acids are changed, or all three are changed. It is preferred to replace Asn at position 56. It is preferred to replace Thr at position 58 with an amino acid other than Ser. It is preferred to not destroy the N-glycosylation site in the CDR2 of the 266 heavy chain by replacing Ser at position 57 with Pro or Asp. Conservative substitutions at one, two, or all three positions are preferred. The most preferred species are those in which Asn at position 56 is replaced with Ser or Thr. Particularly preferred antibodies are those in which Ser or Thr is at position 56, Ser is at position 57, and Thr is at position 58 of SEQ ID NO: 19, SEQ ID NO:20, or SEQ ID NO:21.

The most preferred species are antibodies comprising a light chain of SEQ ID NO:11 and a heavy chain of SEQ ID NO:21, wherein in SEQ ID NO:21, Xaa at position 56 is Ser, Xaa at position 57 is Ser, and Xaa at position 58 is Thr (“N56S”), or wherein in SEQ ID NO:21, Xaa at position 56 is Thr, Xaa at position 57 is Ser, and Xaa at position 58 is Thr (“N56T”).

The preparation of an acceptable pharmaceutical preparation of the antibodies used in the present invention, including its strength, excipients, pH, isotonicity, presentation, dosage form, and the like, is well known to the skilled person. Pharmaceutical compositions for use in the present invention should be appropriate for the selected mode of administration, and pharmaceutically acceptable excipients such as, buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners. Pharmaceutical preparations for use in the present invention should be sterile or at least nearly so, and if necessary preserved or rendered bacteriostatic.

The following example(s) are intended to illustrate, not limit, the invention.

### EXAMPLE 1

Adult, 11 month old females, wild type control and homozygous PDAPP transgenic mice originating from a hybrid genetic background (DBA—C57BL/6—Swiss Webster) [Games et al., Nature. 373:523-527 (1995)], were tested. Approximately 50% of the mice from each genotype group (for sample sizes see tables herein) received 500 μg of mouse monoclonal antibody 266.2 and the other 50% of the mice received phosphate buffered saline (PBS) vehicle administered intra-peritoneally 9 days and 2 days prior to start of behavioral experiments. The behavioral tests were conducted in a fully. randomized and blind manner, i.e. the experimenter had no knowledge of the genotype or the drug treatment history of the subjects. Furthermore, mice were tested in four test chambers so that at any given time one mouse was being tested from each of the four (2 genotypes×2 injection groups) groups. This way any potential circadian changes must have affected all groups in an identical manner.

Prior to testing, and between testing days, animals were group housed (4 mice per cage) in standard plastic cages (32.5×15×15 cm, length×width×depth) with sawdust bedding, and maintained on a 12/12 hr light/dark cycle (lights on at 6 a.m.) with constant temperature (21° C.) and 45% relative humidity. Food and water were available ad libitum.

Four behavioral sessions (6 min each) were conducted over a four-day period as described in detail [Fitch, et al., Hippocampus, 12: 4-17 (2002)]. Briefly, on day one, a habituation session in a neutral context was conducted (behavior was not quantified). This was followed by a training paradigm on day two. Subsequently, on day three, animals were tested in the context memory test and then finally, the cue memory test was conducted on day four. Following each trial, animals were returned to their home cage. Transgenic and wild type mice were randomly assigned to each of four fear conditioning chambers so that 2 transgenic (one treated with the antibody and the other with vehicle) and two wild type mice (one treated with the antibody and the other with vehicle) were running concurrently exactly at the same time. Order of testing was maintained throughout the tests. Each animal was tested in the same chamber in which it was trained. Behavioral experimentation and quantification of data were done blind.

In the neutral context, all animals were exposed to a ‘safe’ environment in which no shocks or tone cues were delivered. This environment was the basic test chamber but visual and tactile contextual cues of the chamber were altered by replacing the shock grid with a perforated acrylic sheet and by installing wall inserts (yellow cartoon paper) inside the cage. Fresh bedding was placed in the drop pan underneath the floor cover of the chamber to provide a familiar (home cage) smell. Between mice the chamber was cleaned with Petzyme (Petsmart, Pacific Coast Distributing, Phoenix, Ariz.). This same ‘neutral context’ was later used for testing tone cue responses. After having been exposed to the neutral context, mice were placed into the test chamber designated ‘unsafe’ where the wall inserts and acrylic floor were removed. Bedding was removed from the drop pan and the chamber and drop pan were cleaned with Steris (St. Louis, Mo.) ‘Coverage Spray’ disinfectant before each subject trial. By using the neutral and unsafe contexts we hoped to facilitate discrimination of relevant contextual cues from other cues, e.g. human handling or cues of the test room, which were always present and which should not be associated with the shock. In the unsafe context (training) subjects were given 10-15 s to acclimatize before behavioral recording was begun. For the first 160 s of the training no stimulation was administered. This adaptation period was followed by a 20 s tone cue (80 db, 3000 Hz), which was co-terminated with a 1 sec 0.7 mA scrambled electric foot shock. This stimulus presentation was repeated at 220 s and again at 280 s for a total of three tone/shock pairings during the 360 s long training session.

The following day, contextual-cues-elicited fear responses were recorded for a 360 s period in the chamber in which training was previously conducted (unsafe context). No tone or shock was presented during this context test. Between individual subject trials, chambers were cleaned with ‘Petzyme’, an agent that provided a novel odor cue. The change in cleaning agents was made in order to minimize the possibility of mice using a salient olfactory cue to identify the unsafe context and thus exhibit their response based on elemental rather than contextual information.

On the final day of the paradigm, elemental tone cue-associated learning was tested in the safe neutral chamber with wall inserts and acrylic floor cover present, and with clean bedding in the drop pan beneath the floor. During this cue test, animals were presented with tone cues identical in amplitude, frequency, and timing of delivery to those given during training, however, no shock was administered. Following each training or test trial, animals were returned to their home cages.

Behavior of the mice was video-recorded with Camcorders (Sony, DCR TRV-20 mini DVCam) and later replayed on a digital VCR (Sony DVCAM DSR-20 digital VCR). Quantification of behavior was conducted using Observer Video Pro software (Noldus, Vageningen, The Netherlands). The software allows the experimenter to quantify predefined motor and posture patterns. The software also makes it possible for the experimenter to control the digital VCR and to synchronize the computer's internal clock with the time stamp generated by the VCR. The following behavioral parameter is quantified: lonz-body (also known in the literature as “stretch attend posture”), hind paws are anchored while the front of the body is moving forward, body is elongated (stretched) and is kept very close to the ground. For the training session the relative duration of long-body was calculated for two intervals, the period preceding the first shock (0-179 sec, neutral acclimation period) and the period including and following the first shock (179-360 sec, period during which subjects experience pain due to electric shock). For the context test, data are expressed for a single interval, the entire period (0-360 sec, period during which subjects experience fear due to presence of contextual stimuli). For the cue test, data are expressed again for two intervals, for the period preceding the first tone cue (0-160 sec, period during which no signs of danger are present) and for the period following this (160-360 sec, a period during which tone cues previously associated with the shock are delivered).

Statistical analyses were conducted using SYSTAT 10 statistical software package on a Compaq PC. Three-way or two-way repeated measures analysis of variance (ANOVA) was conducted to test the effects of genotype (wild type or transgenic), the effects of injection (antibody or vehicle control) and interval, which is the repeated measure factor.

For the training session, significant genotype, antibody, and interval effects were found (Table 1). The interaction terms interval x genotype and interval x genotype x antibody were also significant.

TABLE 1

| | | Relative duration | |
|---|---|---|---|
| | | of Long Body in | |
| | | Training (% ± SEM) | |
| Genotype + Treatment | n | 0-179 sec | 179-360 sec |
| Wild Type + vehicle | 9 | 1.5 ± 0.8 | 34.9 ± 4.1 |
| Wild Type + anti-Aβ antibody | 9 | 0.3 ± 0.3 | 18.3 ± 4.8 |
| PDAPP + vehicle | 12 | 9.6 ± 4.1 | 46.0 ± 5.5 |
| PDAPP + anti-Aβ antibody | 12 | 1.1 ± 0.6 | 43.2 ± 4.7 |

| | | | | | |
|---|---|---|---|---|---|
| Source | SS | df | MS | F | p |

Univariate Repeated Measures Analysis - Between Subjects

| | | | | | |
|---|---|---|---|---|---|
| Genotype | 2581 | 1 | 2581 | 14.1 | 0.001 |
| Antibody | 1076 | 1 | 1076 | 5.86 | 0.020 |
| Genotype *Antibody | 54 | 1 | 54 | 0.294 | 0.591 |
| Error | 6980 | 38 | 184 | | |

Univariate Repeated Measures Analysis - Within Subjects

| | | | | | |
|---|---|---|---|---|---|
| Interval | 21687 | 1 | 21687 | 161 | 0.000 |
| Interval *Genotype | 945 | 1 | 945 | 7.02 | 0.012 |
| Interval *Antibody | 116 | 1 | 116 | 0.86 | 0.360 |
| Interval *Genotype | 576 | 1 | 576 | 4.27 | 0.046 |
| *Antibody | | | | | |
| Error | 5117 | 38 | 135 | | |

Briefly, long-body posture was almost entirely absent before electric shocks were administered, confirming that under baseline condition, i.e. without the presence of pain or fear, mice do not exhibit this behavior. Interestingly, PDAPP transgenic mice did show some appreciable level of long-body posture even during the first period of training when no shocks were given, but this was significantly reduced by the antibody treatment. In response to electric shocks a significant increase of long-body posture is observed in all mice, but this increase was somewhat smaller in wild type animals compared to PDAPP transgenic mice. Furthermore, antibody treated mice, especially the wild type mice, showed a blunted increase of long-body posture.

Similar to the training session, significant antibody effects were observed id the context test and the effect of transgene was also significant (Table 2).

TABLE 2

| | | Relative duration of Long Body in |
|---|---|---|
| | | Context (% ± SEM) |
| Genotype + Treatment | n | 0-360 sec |
| Wild Type + vehicle | 9 | 7.8 ± 2.1 |
| Wild Type + anti-Aβ antibody | 9 | 3.5 ± 1.1 |
| PDAPP + vehicle | 12 | 20.2 ± 3.9 |
| PDAPP + anti-Aβ antibody | 12 | 11.7 ± 2.3 |

Univariate Repeated Measures Analysis - Between Subjects

| Source | SS | df | MS | F | p |
|---|---|---|---|---|---|
| Genotype | 1095 | 1 | 1095 | 13.5 | 0.001 |
| Antibody | 417 | 1 | 417 | 5.14 | 0.029 |
| Genotype *Antibody | 47.8 | 1 | 47.8 | 0.589 | 0.448 |
| Error | 3084 | 38 | 81.2 | | |

The antibody reduced long-body posture in the PDAPP mice almost to the level of the vehicle injected wild type mice. But it is also notable that the antibody had a similar long-body posture reducing effect even when injected in the wild type mice as compared to vehicle injected wild type mice. Variance analysis showed that indeed the effect of the transgene and the effect of the antibody injection did not interact with each other (Table 2, transgene×antibody interaction is non-significant), i.e. the presence of the transgene increased, while antibody injection decreased the amount of long-body posture exhibited.

In the cue test a similar pattern of results was seen but the effects were not statistically significant (Table 3), most probably due to the fact that in this test long-body posture was hardly seen. Nevertheless, the effect of antibody treatment was close to significance (p=0.065).

TABLE 3

| | | Relative duration | |
|---|---|---|---|
| | | of Long Body in | |
| | | Cue test (% ± SEM) | |
| Genotype + Treatment | n | 0-160 sec | 160-360 sec |
| Wild Type + vehicle | 9 | 1.1 ± 0.5 | 1.4 ± 0.8 |
| Wild Type + anti-Aβ antibody | 9 | 0.4 ± 0.4 | 0.7 ± 0.7 |
| PDAPP + vehicle | 12 | 3.1 ± 1.3 | 3.1 ± 1.6 |
| PDAPP + anti-Aβ antibody | 12 | 0.9 ± 0.4 | 1.1 ± 0.5 |

| | | | | | |
|---|---|---|---|---|---|
| Source | SS | df | MS | F | p |

Univariate Repeated Measures Analysis - Between Subjects

| | | | | | |
|---|---|---|---|---|---|
| Genotype | 27.7 | 1 | 27.7 | 2.34 | 0.135 |
| Antibody | 42.7 | 1 | 42.7 | 3.60 | 0.065 |
| Genotype *Antibody | 9.90 | 1 | 9.90 | 0.836 | 0.366 |
| Error | 450 | 38 | 11.8 | | |

Univariate Repeated Measures Analysis - Within Subjects

| | | | | | |
|---|---|---|---|---|---|
| Interval | 0.780 | 1 | 0.780 | 0.113 | 0.739 |
| Interval *Genotype | 0.295 | 1 | 0.295 | 0.042 | 0.837 |
| Interval *Antibody | 0.059 | 1 | 0.059 | 0.009 | 0.927 |
| Interval *Genotype | 0.105 | 1 | 0.105 | 0.015 | 0.903 |
| *Antibody | | | | | |
| Error | 262 | 38 | 6.90 | | |

The fear conditioning paradigm allowed us to investigate shock, contextual stimuli, or tone cue induced behavioral responses. Furthermore, software aided event recording made it possible for us to quantify a posture pattern, long-body, which is associated with fear. This behavior showed a consistent increase in PDAPP mice as compared to wild type control. This increase may be due to Aβ deposition or overexpression of the mutant form of APP in the transgenic mice. Consistent with this, but also surprisingly, our analysis also revealed a significant long-body posture reducing effect of the anti-Aβ antibody treatment.

Note the elevated amount of long-body posture exhibited in response to the three shock-tone pairing during the second half of the training session (3-6 min). This behavior is believed to be the expression of a mild level of anxiety. Also note the consistent reduction of this behavior by antibody treatment in both genotype groups.

Long-body posture has been observed under aversive conditions in mice when cues associated with natural predators are present [Blanchard, et al., Risk assessment and animal models of anxiety. In: Olivier et al. (eds.) Animal models in psychopharmacology. Advances in pharmacological sciences. Basel: Birkhauser Verlag. pp. 117-134 (1991)]. This behavior is also evoked in mice by other fear inducing stimuli including electric shocks or the context in which the shocks were delivered [Fitch, et al. 2002; Gerlai, et al., J. Neuroscience 19:9538-9549 (1999)]. Thus, long-body has been interpreted as a sign of fear [Blanchard, et al. (1991); Fitch, et al. (2002); Gerlai, et al. (1999)]. Accordingly, increased long-body posture may in fact represent increased fear in PDAPP mice compared to wild type control, and decreased long-body posture elicited by the injection of anti-Aβ antibody may represent reduction of fear.

It is important to emphasize that the decrease of long-body posture was observed both in PDAPP mice and also in wild type control. This suggests that perhaps Aβ lowering may have a beneficial, i.e. anxiolytic, effects not only in AD patients but perhaps in the non-demented elderly population as well. Our present results thus raise a new and unexpected possibility: anti-Aβ agents may represent a novel therapeutic application in the treatment of anxiety and related disorders.

1-24. (canceled)

25. A method for treating an anxiety disorder or a mood disorder in an elderly subject, comprising administering to the subject an effective dose of an anti-Aβ antibody:

26. The method of claim 25, wherein the subject is diagnosed or is suspected to have a condition related to Aβ, such as Alzheimer's disease or chronic amyloid angiopathy.

27. The method of claim 25, wherein the subject is not diagnosed or suspected to be suffering from a condition related to Aβ.

28. The method of claim 25, wherein the subject is a human.

29. The method of claim 25, wherein the disorder is a mood disorder.

30. The method of claim 29, wherein the disorder is selected from the group consisting of depression, major depression, minor depression, major depressive episode, and unipolar major depression.

31. The method of claim 29, wherein the disorder is schizophrenia.

32. The method of claim 25, wherein the disorder is an anxiety disorder.

33. The method of claim 32, wherein the disorder is simple phobia.

34. The method of claim 32, wherein the disorder is social phobia.

35. The method of claim 32, wherein the disorder is agoraphobia.

36. The method of claim 32, wherein the disorder is panic disorder.

37. The method of claim 32, wherein the disorder is obsessive-compulsive disorder.

38. The method of claim 32, wherein the disorder is post-traumatic stress disorder.

39. The method of claim 25, wherein the elderly subject is a human whose age is at least 55 years, 60 years, 65, years, 70 years, 75 years, 80 years, 85, years, 90 years, 95 years, 100 years, 105 years, or 110 years.

40. The method of claim 25, wherein the anti-Aβ antibody is a human or humanized antibody.

41. The method of claim 25, wherein the anti-Aβ antibody recognizes or binds to an epitope within between amino acids 1 and 28 of human Aβ.

42. The method of claim 25, wherein the anti-Aβ antibody recognizes or binds to an epitope within between amino acids 13 and 28 of human Aβ.

43. The method of claim 25, wherein the anti-Aβ antibody is selected from the group consisting of 266, N56S, N56T, any antibody comprised of the CDRs of 266, N56S, or N56T, and any antibody that competitively inhibits the binding of 266, N56S, or N56T and human Aβ.

44. The method of claim 42, wherein the anti-Aβ antibody is selected from the group consisting of: an antibody comprised of SEQ ID NO:7 and SEQ ID NO:8; an antibody comprised of SEQ ID NO:9 and SEQ ID NO: 10; an antibody comprised of SEQ ID NO: 11 and SEQ ID NO: 12; an antibody comprised of SEQ ID NO:7 and SEQ ID NO: 19; an antibody comprised of SEQ ID NO:9 and SEQ ID NO:20; and an antibody comprised of SEQ ID NO:11 and SEQ ID NO:21.


SEQUENCE LISTING (21 sequences)

SEQ ID NO: 1 - protein, 16 aa, Mus sp.
RSSQSLIYSDGNAYLH

SEQ ID NO: 2 - protein, 7 aa, Mus sp.
KVSNRFS

SEQ ID NO: 3 - protein, 9 aa, Mus sp.
SQSTHVPWT

SEQ ID NO: 4 - protein, 5 aa, Mus sp.
RYSMS

SEQ ID NO: 5 - protein, 17 aa, Mus sp.
QINSVGNSTYYPDTVKG

SEQ ID NO: 6 - protein, 3 aa, Mus sp.
GDY

SEQ ID NO: 7 - protein, 113 aa, Artificial Sequence
  synthetic construct - humanized
DXVMTQXPLSLPVXXGQPASISCRSSQSLXYSDGNAYLHWFLQKPGQSPXLLIYKVSNRFSGVPDRFSGSGSGTDFTLKISRVEAEDXGVYYCSQSTHVPWTFGXGTXXEIKR

SEQ ID NO: 8 - protein, 112 aa, Artificial Sequence
  synthetic construct - humanized
XVQLVEXGGGLVQPGGSLRLSCAASGFTFSRYSMSWVRQAPGKGLXLVAQINSVGNSTYYPDXVKGRFTISRDNXXNTLYLQMNSLRAXDTAVYYCASGDYWGQGTXVTVSS

SEQ ID NO: 9 - protein, 113 aa, Artificial
  Synthetic construct - humanized
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 10 - protein, 112 aa, Artificial
  Synthetic construct - humanized
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 11 - protein, 219 aa, Artificial
  Synthetic construct - humanized
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 442 aa, Artificial
  Synthetic construct - humanized
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 17 aa, Mus sp.
  MISC_FEATURE (7)..(7): Xaa at position 7 is any amino acid, provided that if Xaa at position 8 is neither Asp nor Pro and Xaa at position 9 is Ser or Thr, then Xaa at position 7 is not Asn
QINSVGXXXYYPDTVKG

SEQ ID NO: 14 - protein, 17 aa, Mus sp.
  MISC_FEATURE (7)..(7): Xaa at position 7 is selected from the group consisting of Ala, Cys, Asp, Glu, Phe, Gly, His, Ile, Lys, Leu, Met, Pro, Gln, Arg, Ser, Thr, Val, Trp, and Tyr
QINSVGXXXYYPDTVKG

SEQ ID NO: 15 - protein, 17 aa, Mus sp.
  MISC_FEATURE (7)..(7): Xaa=Asn
QINSVGXXXYYPDTVKG

SEQ ID NO: 16 - protein, 17 aa, Mus sp.
  MISC_FEATURE (7)..(7): Xaa=Asn
QINSVGXXXYYPDTVKG

SEQ ID NO: 17 - protein, 17 aa, Mus sp.
  MISC_FEATURE (7)..(7): Xaa at position 7 is selected from the group consisting of Ala, Gly, His, Gln, Ser, and Thr
QINSVGXXXYYPDTVKG

SEQ ID NO: 18 - protein, 17 aa, Mus sp.
  MISC_FEATURE (7)..(7): Xaa at position 7 is Asn
QINSVGXXXYYPDTVKG

SEQ ID NO: 19 - protein, 112 aa, Artificial
  synthetic construct - humanized
XVQLVEXGGGLVQPGGSLRLSCAASGFTFSRYSMSWVRQAPGKGLXLVAQINSVGXXXYYPDXVKGRFTISRDNXXNTLYLQMNSLRAXDTAVYYCASGDYWGQGTXVTVSS

SEQ ID NO: 20 - protein, 112 aa, Artificial
  synthetic construct - humanized
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSS

SEQ ID NO: 21 - protein, 442 aa, Artificial
  synthetic construct - humanized
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK